(19) United States
(12) Patent Application Publication
(10) Pub. No.: US 2010/0145182 A1
(43) Pub. Date: Jun. 10, 2010

(54) METHOD TO CONTROL THE ACQUISITION OPERATION OF A MAGNETIC RESONANCE DEVICE IN THE ACQUISITION OF MAGNETIC RESONANCE DATA OF A PATIENT, AND ASSOCIATED MAGNETIC RESONANCE DEVICE

(76) Inventors: Michaela Schmidt, Uttenreuth (DE); Peter Speier, Erlangen (DE); Stefan Assmann, Erlangen (DE); Sebastian Schmidt, Weisendorf (DE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/631,084

(22) Filed: Dec. 4, 2009

(30) Foreign Application Priority Data
Dec. 5, 2008 (DE) 10 2008 060 719.3

(51) Int. Cl. A61B 5/055; G06Q 50/00; G06F 3/048; G01R 33/48

(52) U.S. Cl. 600/410; 705/2; 715/810; 324/309

(57) ABSTRACT

In a method to control the acquisition operation of a magnetic resonance device in the acquisition of magnetic resonance data of a patient, and associated magnetic resonance device, patient-related acquisition parameters are determined, technical control parameters are automatically determined taking into account the patient-related acquisition parameters, and the magnetic resonance device is controlled according to the control parameters.

### BACKGROUND OF THE INVENTION

1. Field of the Invention

The present invention concerns a method to control the data acquisition operation of a magnetic resonance device in the acquisition of magnetic resonance data of a patient, as well as a corresponding magnetic resonance device.

2. Description of the Prior Art

Magnetic resonance imaging is by now an established imaging method that has the benefit of making a large number of imaging possibilities, but this benefit entails the difficulty of finding the optimal settings for an optimal diagnostic image quality. In addition to the specific, planned diagnosis, the physical-technical control parameters of the magnetic resonance device that are required for high quality image acquisition are also dependent on the specific physiology of the patient to be examined.

It follows from this that the operation of a magnetic resonance device is very complex and, for medical personnel, anything but self-explanatory due to the number of technical-physical control parameters that must be set. Due to a lack of understanding of the technical parameters, results of poor or even unusable quality will occur due to incorrect parameter selection. Particularly complicated examinations (for example the depiction of the beating heart) require the adaptation of many technical control parameters to the patient physiology in order to acquire diagnostically evaluable image data.

Currently, only well-trained personnel can thus conduct magnetic resonance examinations. Instruction in magnetic resonance imaging can take a very long time, for example three to six months. It is necessary to have at least a basic understanding of the physics underlying magnetic resonance in order to be able to choose the correct control parameter settings. The adaptation of the examination to the physiology of the patient through technical control parameters requires time and substantiated basic knowledge.

### SUMMARY OF THE INVENTION

An object of the present invention is to provide a simpler, faster method to control the acquisition operation of a magnetic resonance device, which is particularly suitable for less trained personnel.

This object is achieved by a method in accordance with the invention wherein patient-related acquisition parameters are determined, technical control parameters are determined taking into account the patient-related acquisition parameters, and the magnetic resonance device is controlled according to the control parameters.

While the control parameters must be directly determined (in particular input) in the prior art, the present invention determines patient-related acquisition parameters that are better accessible and understandable to medical personnel, in particular via input, these patient-related acquisition parameters describing the physiology of the patient to whom the acquisition process should be adapted, this physiology being relevant to the magnetic resonance imaging. With consideration of these acquisition pars, control parameters for the magnetic resonance device that represent the technical-physical translation of the patient-related acquisition parameters are then automatically determined without further action of the user. As used herein, determination of the control parameters naturally also encompasses an adaptation of already-present control parameters, for example those set to standard values. The magnetic resonance device is controlled according to the control parameters in order to acquire image data of the patient. In this way a significant simplification and acceleration of the adjustment of the magnetic resonance device to the specific physiology of the current patient can be conducted independent of the type of determination of the patient-related acquisition parameters. This is particularly true since frequently one patient-related acquisition parameter can already (concurrently) determine the values of multiple control parameters. However, the input of complex physical-technical control parameters is substantially reduced. An improved image quality can thus ultimately be achieved simply and quickly for every patient.

As mentioned, the term “determination of control parameters under consideration of the patient-related parameters” is to be understood broadly. In particular, an existing measurement protocol embodying a magnetic resonance sequence is adapted and/or a new measurement protocol embodying a magnetic resonance sequence is selected to determine the control parameters. Such basic known measurement protocols frequently also include the acquisition technique that should be used to acquire the image data. Such a measurement protocol, which ultimately represents a synopsis of at least a portion of the control parameters, can also be an organ program (for example) which can then be adapted using the acquisition parameters. Arbitrary possibilities are conceivable.

An acquisition technique and/or control parameters describing a sequence and/or sequence-specific control parameters (in particular the flow speed in functional magnetic resonance imaging) and/or control parameters describing the slice geometry and/or control parameters describing the contrast (in particular the bandwidth and/or a TR value and/or the resolution) and/or acceleration parameters (in particular with regard to a fast Fourier transformation) and/or post-processing parameters and/or evaluation parameters can be determined as control parameters. This enumeration is not to be considered as conclusive, but rather indicates using examples that a control parameter can ultimately relate to any functionality of the magnetic resonance device.

In a preferred embodiment of the present invention, at least a portion of the acquisition parameters (in particular all acquisition parameters) can be input by a user (in particular via a user interface). In this case, the method according to the invention has a few additional advantages. User interfaces can be of fundamentally simpler design so that they are also accessible to users who do not have the elaborate preparatory training. With the aid of such intelligible and patient-oriented acquisition parameters, the instruction time for the operator in magnetic resonance imaging can be markedly reduced, or less qualified persons can also operate the magnetic resonance device. However, in this embodiment an additional advantage is provided because it can flexibly address the current state of the patient. Data can thus also be taken into account that cannot be determined automatically.

Acquisition parameters describing the current state of the patient—in particular the patient's willingness to cooperate and/or the patient's ability to hold his breath and/or the patient's physical restiveness—are input before and/or during the acquisition process. It is consequently possible in a simple manner to take into consideration particular current states of the patient, for example his willingness to cooperate with the personnel or his physical restiveness, this how still he will lie in the patient receptacle. For example, it can be provided that an image acquisition technique optimized for increased patient movement (in particular a BLADE technique) and/or activation parameters determining a lower resolution are selected given a restless and/or uncooperative patient. In particular, the simple specification of the information of whether the patient is restless and/or cooperative is already sufficient instead of having to laboriously reconsider all physical-technical control parameters given a patient who is not to be immobilized. This state can also change during the acquisition operation; for example, patients can become restless or the like with time due to constriction in the patient receptacle. This can be flexibly reacted to—thus during data acquisition in examinations comprising multiple image acquisitions, for example—and the image acquisition technique can be changed, for example.

With regard to the breath hold time of the patient, control parameters of a segmented measurement (in particular the number of segments and/or an acceleration factor and/or a matrix size) and/or control parameters of a multi-slice measurement (in particular the number of slices per breath hold phase) are determined given an acquisition parameter reflecting the possible breath hold time of the patient, and/or a new measurement technique (in particular a real time measurement and/or a measurement with the BLADE technique) are selected given a breath hold time that is too short and/or if a breath hold is not possible.

Many measurements in the torso region should be implemented either over an entire cardiac cycle or at a specific phase of the cardiac cycle, which means that the time window is frequently very small, such that the goals of measuring with optimally high resolution and as free of movement artifacts as possible cannot be optimally achieved simultaneously, such that a plurality of acquisition techniques whose suitability depends on the concrete patient are known in this field. For example, a segmented measurement is known in which only a few image lines are measured at high resolution at a specific cardiac phase or breathing phase (for example are triggered) so that the entire image results over multiple cardiac cycles. Such triggering can also be provided in multi-slice measurements. In order to not have problems with the breathing cycle, in such cases it is typical to have the patient hold his breath in order to then conduct as many measurements as possible during this breath hold time. If it is not possible for the patient to hold his breath for physiological reasons, or if he can only hold his breath for too short a period of time, in particular the segmented measurement cannot be implemented; rather, measurement must be made during a cardiac phase. However, if this is known, the measurement technique and the measurement type can be accordingly adapted. A real time measurement can be implemented (thus a contiguous measurement), or what is known as the BLADE technique (which is particularly insensitive to movement) can be used. A reorganization in k-space is thereby provided. The BLADE technique is frequently also designated as a PROPELLER technique (periodically rotated overlapping parallel lines with enhanced reconstruction). In addition to the current unrest of the patient, such a breath hold time is also frequently a parameter describing the current state of the patient.

In the present invention it is also possible that, in addition to the patient-related acquisition parameters, additional (in particular abstract) acquisition parameters are also input and taken into account in the determination of the activation parameters. For example, a focal point can be placed by a user at a user interface as to whether the user has a greater requirement for a high resolution image or would rather seek a fast image acquisition. It is not direct control parameters that are input, but rather communication with the user on an abstract, easily understandable basis.

In a further embodiment for parameter input, in the input of multiple acquisition parameters and/or given another previous determination of at least one acquisition parameter, the already-known acquisition parameters are evaluated to determine a suggestion for at least one acquisition parameter and/or control parameter that is still to be input, and this suggestion is displayed to a user. The selection of possibilities for current acquisition parameters to be input can already be limited by known acquisition parameters, and a most probable input can also be concluded. The interactive user direction can be further improved and intelligibly presented in this way. In the simplest case, the user has only to confirm the suggestion.

As a whole, in the event that at least a portion of the acquisition parameters are input by a user via a user interface, a particularly simple user direction that is also accessible to personnel less trained in magnetic resonance imaging can thus be realized, so the process is simplified and accelerated.

It should be noted that the capability can be provided in the user interface to allow a user to switch to a mode where the user can directly input the physical-technical control parameters as is customary in the prior art. The user interface can then be configured as needed for different users.

Alternatively, or preferably in addition to the input of the acquisition parameters by a user, at least a portion of the acquisition parameters are automatically determined, in particular by querying data from an examination apparatus and/or a patient monitoring apparatus and/or a hospital information system and/or an electronic patient file. Some information (in particular such information that does not pertain to the current state of the patient) is already present as diagnoses or other patient data and can advantageously be retrieved from a hospital information system and/or an electronic patient file so that it does not need to be input any more. Another source of patient-specific information is examination apparatuses and/or patient monitoring apparatuses. Such apparatuses that measure the physiological properties of the patient are to some extent part of the magnetic resonance device itself or are arranged in proximity to it and involved in a communication connection with it. EKG and blood pressure measurement devices, spirographs or the like are cited as examples. An additional simplification and acceleration is also enabled in this way.

For example, a different acquisition technique is selected based on data of a patient monitoring device determining the respiratory activity (in particular the oxygen saturation) of the patient, in particular a technique that works without the patient holding his breath given a low oxygen saturation. Here the aforementioned segmentation multi-slice acquisition, real time or BLADE techniques can be cited. A spirogram of the patient is retrieved from a hospital information system and/or an electronic patient file and is in particular evaluated with regard to the ability of the patient to hold his breath. In particular the breath-hold time can be determined or estimated from this, whereupon the conclusions already described above with regard to the manual input can be drawn with regard to the control parameters. In particular, it is also conceivable to present such an estimated breath-hold time as a suggestion given an input of the user.

For example, if the spirometry yields as a result a vital capacity of two liters, the breath-hold time can be automatically set to a minimum and in particular be proposed to the user in the user interface. Other parameters of heart-lung diagnostics that can be retrieved from a hospital information system and/or an electronic patient file can also be appropriately taken into account, for example the breathing rate or the ejection fraction.

Furthermore, ICD-coded diagnoses and/or EKG data can be retrieved from a hospital information system and/or an electronic patient file and be evaluated with regard to acquisition parameters and/or control parameters to be input. These do not have to be diagnoses related to the current magnetic resonance acquisitions, such that R00 numbers (disruption of the heart beat) are generally assessed as signs of arrhythmias in the cardiac cycle for measurement in the torso region that possibly make triggered measurement methods impossible. Additional diagnoses that are relevant to magnetic resonance imaging are, for example, lung diseases that allow only short breathing pauses or none at all, or mental or neurological illnesses given which cooperation of the patient is not to be expected, and therefore a movement correction or a corresponding technique (for example the BLADE technique) will be necessary. EKG data that can be retrieved from a hospital information system (for example) can provide an indication of arrhythmias or other relevant abnormalities. For example, if the pulse rate typically exceeds a limit value, a simpler measurement protocol or a simpler measurement technique can be selected, in particular without an EKG triggering and without a breath hold. Previously acquired EKG data can particularly advantageously be tentatively be assigned as input values to an algorithm used for triggering. If the algorithm does not succeed in extracting a reasonable trigger information from these, an untriggered measurement protocol is automatically used.

Furthermore, data regarding kidney function can be queried from a hospital information system and/or an electronic patient file and are evaluated in particular with regard to a contrast agent amount to be administered. An example of such data is creatinine or urea values, or also the glomerular filtration rate. These can be used in order to calculate the contrast agent amount or also in order to suggest a corresponding measurement protocol that works with less or without contrast agent.

In a further embodiment, at least one suggestion for an acquisition parameter to be input by a user is determined from the automatically determined acquisition parameters and is displayed to the user at the input. In particular, data that can be automatically provided can be used in order to estimate beforehand what the current state of the patient could be. However, suggestions can also be generally made. For example, if it is known from an electronic patient file that the patient has arrhythmias in the cardiac cycle, in principle acquisition techniques can be proposed that work without triggering or the like.

It can generally be provided that the number of measurements for a multi-slice measurement is determined in the acquisition parameters describing the patient size. For example, this is relevant if a whole body magnetic resonance imaging or a pelvis-leg angiography should occur. The patient size then determines the how many measurements are required in order to be able to acquire the entire region of interest.

Given an acquisition parameter displaying an arrhythmia in the cardiac cycle of the patient and a provided segmented measurement, it can be provided that a real-time measurement is chosen instead of this. Namely, in most cases it is then not possible or is only possible with difficulty to produce a reasonable triggering.

Given an acquisition parameter indicating the nationality of the patient, it can be additionally provided that an instruction output device, in particular a device to automatically output an instruction related to the breathing of the patient, is activated in the corresponding language. This means that the announcements for the patient can occur in his native language and thus are equally understandable. An instruction output device is most often realized in the form of headphones. Announcements for various languages are stored in an associated control device, wherein the acquisition parameter is converted into a corresponding control parameter that chooses the corresponding language.

As already mentioned, it can be provided that, despite the availability of automated determination control parameters can be directly input by a user. This can in particular occur according to the selection of a corresponding option by a user, in particular in a user interface. In particular, an option can be provided that enables the user to entirely input only the physical-technical control parameters, as this is known in the prior art.

In addition to the method, the invention also concerns a magnetic resonance device with a control apparatus that is fashioned to implement the method according to the invention in one of the cited embodiments. In particular, a user interface reproducible on a display device can be provided for the input of at least a part of the acquisition parameters by a user. The control apparatus is accordingly fashioned to receive and/or query patient-related acquisition parameters via an input device, for example from an examination apparatus and/or a patient monitoring apparatus and/or a hospital information system and/or an electronic patient file. These patient-related acquisition parameters are then taken into account as described by the control apparatus in the automatic determination of control parameters. The control apparatus is also fashioned to control the magnetic resonance device corresponding to the determined control parameters. By the use of patient-related (thus patient-centric) acquisition parameters it is possible to provide the magnetic resonance device with a user interface which also allows a less technically/physically trained user to make the necessary adjustments in a simple and quick manner. In this way the acceptance by the user is increased. In a particular embodiment, the user interface is fashioned to switch to the input of at least one control parameter by a user in a manner controlled by the user. This means that a particularly experienced user can furthermore directly transfer the physiology of the patient into suitable control parameters.

### BRIEF DESCRIPTION OF THE DRAWINGS

FIG. 1 shows the basic workflow of an embodiment of the method according to the invention.

FIG. 2 schematically illustrates a magnetic resonance device according to the invention.

### DESCRIPTION OF THE PREFERRED EMBODIMENTS

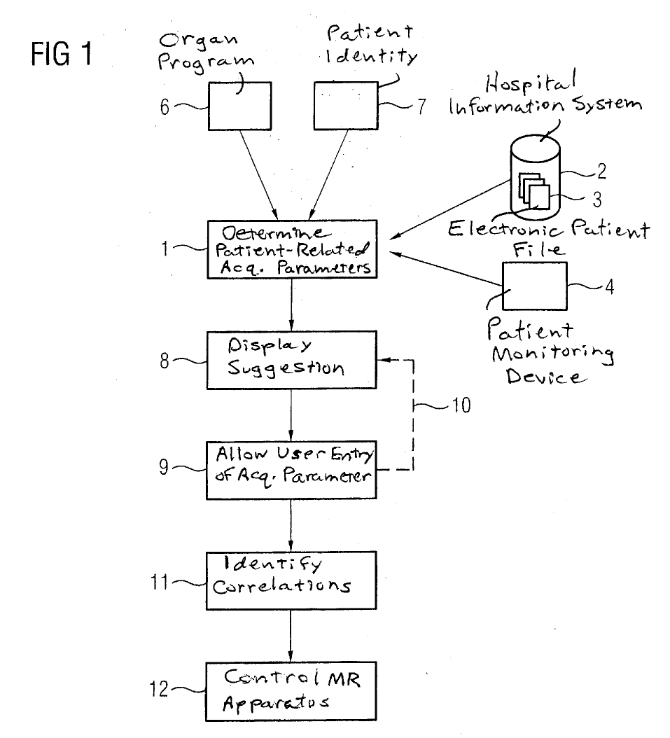

FIG. 1 shows a principle drawing that explains in detail the workflow of the method according to the invention. According to the method according to the invention, physical-technical control parameters are no longer input as has been typical in order to enable a magnetic resonance imaging; rather, patient-related acquisition parameters are determined that are input at least in part by a user.

The method according to the invention begins in Step 1. In this, patient-related acquisition parameters relevant to an magnetic resonance imaging task are retrieved from various external sources (presently from a hospital information system 2), are stored in the electronic patient file 3, and [sic] to a patient monitoring device 4.

In the following it should be assumed that two basic items of information are already present, namely an organ program 6 which essentially specifies which diagnostic goal the magnetic resonance acquisitions are pursuing and in particular contains prefabricated control parameters, and the identity 7 of the patient who should be examined.

After a portion of the patient-related acquisition parameters have already been determined in this way in Step 1, additional patient-related acquisition parameters and possibly also abstract additional acquisition parameters should now be input by a user via a user interface. For this it is initially provided in Step 8 to conclude suggestions for acquisition parameters that are still to be input in a pre-evaluation from the acquisition parameters that are already known. This is optional and in particular also does not need to ensue for every acquisition parameter that is still to be input.

At least one additional acquisition parameter is then input in Step 9 by a user via a user interface. At this point it is noted that naturally suggestions for acquisition parameters can in turn be determined in Step 8 with the database that is expanded in this way, as this is represented by the dashed arrow 10. The suggestions that are present are displayed in the user interface and can be adopted by a user via a simple confirmation.

In particular, acquisition parameters related to the current state of the patient that cannot be determined or predicted automatically in Step 1 without further measures from the databases that are already present can also be input in Step 9. The cooperativeness of the patient, his ability to hold his breath and his physical unrest are cited merely as examples. In particular, it can be provided that this input of acquisition parameters related to the current state of the patient can also ensue directly before or during the acquisition process, such that there can be a flexible reaction to altered circumstances.

Abstract additional acquisition parameters that are not directly related to the patient can also be input in Step 9; requirements are thus posed in a generally intelligible form via the user interface. For example, it can be queried whether a particularly quick or a particularly high-resolution acquisition is preferred, which then can be qualitatively determined by the user as an acquisition parameter, for example via a scale or the like.

A determination of technical control parameters is now automatically implemented using a control apparatus of the magnetic resonance device under consideration of the patient-related acquisition parameters and the additional abstract acquisition parameters. A measurement protocol predetermined by the organ program can thereby be adapted; however, it is also possible—depending on the acquisition parameters—that a completely new measurement protocol is selected. Control parameters that are normally not contained in the measurement protocol can be determined and/or adapted. These are explained again in detail in the following using a few examples. For example, it can thus be provided that—if the acquisition parameters indicate a restless or uncooperative patient—an image acquisition technique optimized for an increased patient movement is switched to. An example of this is what is known as the BLADE technique. A lower resolution and thus a faster data acquisition can also be additionally selected in this case. In another example, the possible breath-hold time of the patient can be analyzed as an acquisition parameter. Control parameters can thereby be adapted to a segmented measurement, for example the number of segments; however, control parameters can also be determined in a multi-slice measurement, in particular the number of slices per breath-hold phase. If the breath-hold time is too short or a breath-hold is not even possible in the first place, a different measurement technique can even be chosen, for example in the form of a different protocol; a real-time measurement can be implemented, for example. In this context it is noted that a possible inability of the patient to hold his breath can already be concluded from the acquisition parameters automatically retrieved in Step 1, for example if data of a patient monitoring device 4 monitoring the respiratory capability of the patient are queried, or if a spirogram of the patient is stored in a hospital information system 2 or an electronic patient file 3. Given an oxygen saturation, of only 90%, for example, a technique can be switched to that manages without the patient holding his breath. Given a vital capacity of, for example, two liters, the possible breath-hold time would also automatically be set to a minimum. It is noted that this can naturally also first be indicated in Step 8 as a suggestion that is then only confirmed by the user in Step 9.

Furthermore, ICD-coded diagnoses or EKG data (for example) can also be obtained from the hospital information system 2 or the patient file 3. The presence of arrhythmias in the cardiac cycle or other relevant properties of the patient (for example lung diseases or neurological diseases) can be concluded from these that would likewise require deviating acquisition techniques or, respectively, deviating control parameters.

Finally, data regarding kidney function are to be cited, for example creatinine or urea values or also the glomerular filtration rate, which can have an effect on the possible contrast agent amount that is to be used.

The patient size can allow an improvement of multi-slice acquisitions—for example of whole-body magnetic resonance imaging or pelvis-leg angiography—with regard to the number of measurements; the presence of arrhythmias in the cardiac cycle can indicate that a triggering must be adapted, or triggered measurements such as segmented measurements are even completely impossible. Even if particularly simple examples have ultimately been cited here for explanatory purposes, the consideration of arbitrarily complex correlations in Step 11 is also possible in the derivation of the control parameters under consideration of the acquisition parameters.

Control parameters of the magnetic resonance device that are additionally connected with the acquisition activity can also be determined, for example control parameters for an instruction output device (for example headphones). The nationality of the patient can thus define as acquisition parameters control parameters that ensure that the instruction is output in the correct language.

After the control parameters have been determined in Step 11, in Step 12 the magnetic resonance device is automatically controlled correspondingly so that image improved image data adapted to the current patient can be generated.

An option that allows one or more control parameters to also be directly input in the event that this is desired by a user can also be present in the user interface.

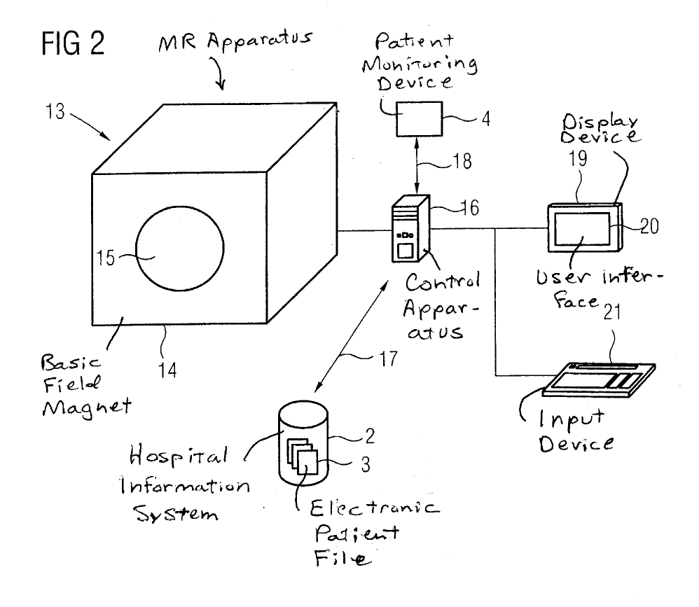

FIG. 2 shows a magnetic resonance apparatus 13 according to the invention which comprises a basic field magnet 14 with a patient receptacle 15 into which a patient can be inserted for examination. The basic functionality of magnetic resonance devices and their concrete embodiment is widely known in the prior art and does not need to be presented in detail herein, which is why only components that are relevant to the invention are shown here. The magnetic resonance device 13 thus comprises a control apparatus 16 that is fashioned to execute the method according to the invention, thus to determine patient-related acquisition parameters; to automatically determine technical control parameters under consideration of the patient-related acquisition parameters; and to control the magnetic resonance device 13 according to the control parameters. For this purpose, a communication connection 17 exists between the control apparatus 16 and the hospital information system 2 with the electronic patient file 3, and a communication connection 18 exists with the patient monitoring apparatus 4. Furthermore, a display device 19 is provided on which a user interface 20 as described above can be displayed. An input of acquisition parameters or also other parameters is possible via an input device 21.

The user interface 20 can be fashioned for user-controlled switching to the input of at least one control parameter by a user, such that ultimately a flexibility also exists to the extent of how the personal preferences of the user appear with regard to the configuration of the magnetic resonance device 13.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for controlling an acquisition operation to acquire magnetic resonance data with a magnetic resonance apparatus, comprising the steps of:
for a patient from whom magnetic resonance data are to be acquired with a magnetic resonance apparatus, determining patient-related acquisition parameters that have an effect on the acquisition of said magnetic resonance data from the patient in the magnetic resonance apparatus;
providing said patient-related acquisition parameters to a processor and, in said processor, automatically determining technical control parameters dependent on said patient-related acquisition parameters; and
from said processor, controlling operation of said magnetic resonance apparatus according to said control parameters to obtain a set of magnetic resonance data from the patient.

2. A method as claimed in claim 1 wherein the step of providing said patient-related acquisition parameters to a processor comprises manually entering, through a user interface connected to said processor, at least a portion of said patient-related acquisition parameters.

3. A method as claimed in claim 2 comprising selecting patient-related acquisition parameters, in said portion of said patient-related acquisition parameters that are manually entered through said user interface, from the group consisting of acquisition parameters describing a willingness of the patient to cooperate in acquiring said magnetic resonance data, and ability of the patient to implement a breath hold during acquisition of said magnetic resonance data, and physical restiveness of the patient.

4. A method as claimed in claim 3 comprising, when said patient-related parameters indicate an uncooperative or restless patient, automatically determining, in said processor, technical control parameters that accommodate patient movement or cause said magnetic resonance dataset to be acquired with a lower resolution.

5. A method as claimed in claim 3 comprising, when said patient-related parameters indicate the ability of the patient to implement a breath hold, automatically determining said technical control parameters in said processor from a group consisting of a number of segments in a segmented acquisition of said set of magnetic resonance data, an acceleration factor in a segmented acquisition of said set of magnetic resonance data, a matrix size in a segmented acquisition of said set of magnetic resonance data, a number of slices per breath hold in a multi-slice acquisition of said set of magnetic resonance data, a real time acquisition of said set of magnetic resonance data, and acquisition of said set of magnetic resonance data with the BLADE technique.

6. A method as claimed in claim 2 comprising, in addition to said portion of said patient-related parameters entered into said processor through said user interface, manually entering additional parameters into said processor through said user interface that quantify a preference of the user that have an effect on said acquisition of said set of magnetic resonance data with said magnetic resonance apparatus.

7. A method as claimed in claim 6 comprising, as said additional parameters, entering additional parameters selected from the group consisting of a speed of said acquisition of said set of magnetic resonance data and a resolution of said set of magnetic resonance data, into said processor through said user interface.

8. A method as claimed in claim 2 comprising, in said processor, automatically generating a suggestion for at least one acquisition parameter or control parameter not yet entered into said processor, and displaying said suggestion at said user interface.

9. A method as claimed in claim 1 wherein the step of said determining patient-related acquisition parameters comprises automatically determining at least a portion of said patient-related acquisition parameters from a source selected from the group consisting of data acquired from an examination apparatus that interacts with said patient, data acquired from a patient monitoring apparatus that interacts with said patient, data stored in a hospital information system, and data stored in an electronic patient file for said patient.

10. A method as claimed in claim 1 wherein the step of determining patient-related acquisition parameters comprises determining a parameter indicative of respiratory activity of the patient, and wherein the step of automatically determining technical parameters comprises determining oxygen saturation of the patient from said respiratory activity and, given a low oxygen saturation, selecting, as said control parameters, a technique for acquiring said set of magnetic resonance data that does not require the patient to implement a breath hold.

11. A method as claimed in claim 10 comprising determining said respiratory activity from a spirogram of the patient obtained from a source selected from the group consisting of a hospital information system and an electronic patient file of the patient.

12. A method as claimed in claim 1 wherein the step of determining said patient-related acquisition parameters comprises retrieving patient-related parameters selected from the group consisting of ICD-coded diagnoses and ECG data, from a source selected from the group consisting of a hospital information system and an electronic patient file of the patient.

13. A method as claimed in claim 1 wherein the step of determining patient-related acquisition parameters comprises determining data regarding kidney function of the patient from a source selected from the group consisting of a hospital information system and an electronic patient file of the patient and comprising automatically determining said technical control parameters as control parameters that define an amount of contrast agent to be administered to the patient dependent on said kidney function.

14. A method as claimed in claim 1 comprising determining patient-related acquisition parameters that indicate a susceptibility of the patient to a physical risk during said acquisition of said set of magnetic resonance data and, when said risk is determined by said processor to be present, automatically providing a suggestion from said processor, via a user interface connected to the processor, for at least one acquisition parameter or control parameter that is yet to be entered into said processor.

15. A method as claimed in claim 1 comprising determining patient size as one of said patient-related acquisition parameters and, in said processor, automatically determining a number of slices for a multi-slice acquisition of said set of magnetic resonance data, as one of said control parameters.

16. A method as claimed in claim 1 comprising determining, as one of said patient-related parameters, a parameter indicative of an arrhythmia in a cardiac cycle of the patient and, if said arrhythmia is determined by said processor to be present, automatically selecting a real-time acquisition of said set of magnetic resonance data in said processor as said control parameters.

17. A method as claimed in claim 1 comprising determining, as one of said patient-related parameters, a nationality of the patient and, from said processor, providing humanly perceptible instructions to the patient, as one of said control parameters, via a patient instruction device, in a language dependent on said nationality.

18. A method as claimed in claim 1 comprising providing a user interface to said processor and, through said user interface, allowing selection of an option by a user to enter a user-selected control parameter into said processor and, after selection of said option, directly entering said user-selected control parameter into said processor.

19. A method as claimed in claim 1 comprising, in said processor, determining said control parameters by adapting a previously-implemented magnetic resonance sequence dependent on said patient-related parameters, or selecting a new data acquisition protocol formulated as a magnetic resonance sequence dependent on said patient-related acquisition parameters.

20. A method as claimed in claim 1 comprising selecting said control parameters from the root consisting of an acquisition technique for acquiring said set of magnetic resonance data, a magnetic resonance pulse sequence for acquiring said set of magnetic resonance data, control parameters that are specific to a predetermined magnetic resonance data sequence, flow speed in a functional magnetic resonance imaging sequence, control parameters describing slice geometry, control parameters describing contrast, control parameters describing a bandwidth, control parameters describing a TR value, control parameters describing a resolution of said data, control parameters describing an acceleration factor, control parameters describing implementation of a fast Fourier transformation, post-processing parameters, and evaluation parameters.

21. A magnetic resonance apparatus comprising:
a magnetic resonance data acquisition device configured to receive a patient therein to acquire magnetic resonance data therefrom;
a computerized control unit in communication with said magnetic resonance data acquisition device;
an input port allowing entry into said control unit of patient-related acquisition parameters that have an effect on a manner of acquiring said magnetic resonance data from the patient with the magnetic resonance data acquisition unit; and
said computerized control unit being configured to automatically determine control parameters, dependent on said patient-related acquisition parameters, for operating said magnetic resonance data acquisition unit to acquire said magnetic resonance data from the patient, and to operate said magnetic resonance data acquisition unit with said control parameters to acquire a set of magnetic resonance data from the patient.

22. A magnetic resonance apparatus as claimed in claim 21 comprising a user interface connected to said computerized control unit that allows manual entry by a user into the processor of at least one of said patient-related acquisition parameters.

23. A magnetic resonance apparatus as claimed in claim 22 wherein said user interface is configured to allow manual entry by a user of an additional acquisition parameter that indicates a preference of the user for operating said magnetic resonance data acquisition unit to acquire said set of magnetic resonance data from the patient.

24. A magnetic resonance apparatus as claimed in claim 22 wherein said user interface is configured to allow direct entry of a user-selected control parameter into said processor.
